(19) United States
(12) Patent Application Publication
(10) Pub. No.: US 2022/0379069 A1
(43) Pub. Date: Dec. 1, 2022

(54) ONE-WAY RESPIRATORY VALVE AND ANIMAL ASSISTIVE DRUG DELIVERY RESPIRATOR PROVIDED WITH ONE-WAY RESPIRATORY VALVE

(71) Applicant: Wenzen Health (Shanghai) Co.,Ltd, Shanghai (CN)

(72) Inventors: Zheyu Ding, Shanghai (CN); Ya Shao, Shanghai (CN)

(73) Assignee: Wenzen Health (Shanghai) Co.,Ltd, Shanghai (CN)

(21) Appl. No.: 17/504,549

(22) Filed: Oct. 19, 2021

(30) Foreign Application Priority Data
May 25, 2021 (CN) 202110570201.5

(51) Int. Cl. A61M 16/20; A61M 16/06

(52) U.S. Cl. CPC A61M 16/208; A61M 16/06; A61M 2250/00

(57) ABSTRACT

A one-way respiratory valve includes a valve body. The valve body is hollow, opposite ends of the valve body are open, a partition plate is provided at one of the opposite ends, and an inhalation hole is formed at the other one of the opposite ends; an exhalation hole is formed on a side wall of the valve body, a second unidirectional guide member is provided at the exhalation hole of the valve body, and the second unidirectional guide member is unidirectionally accessible from an inside of the valve body to an outside of the valve body; and a middle part of the partition plate protrudes towards the inhalation hole to form a first protrusion, a first unidirectional guide member is provided on the first protrusion, and the first unidirectional guide member is unidirectionally accessible from the outside of the valve body to the inside of the valve body.

### CROSS REFERENCE TO THE RELATED APPLICATIONS

This application is based upon and claims priority to Chinese Patent Application No. 202110570201.5, filed on May 25, 2021, the entire contents of which are incorporated herein by reference.

### TECHNICAL FIELD

This application relates to the field of assistive drug delivery devices, and in particular to a one-way respiratory valve and an animal assistive drug delivery respirator provided with the one-way respiratory valve.

### BACKGROUND

An animal assistive drug delivery respirator is specially designed for any animal that has a respiratory tract and needs inhalation drug therapy. The animal assistive drug delivery respirator can store a drug and facilitate the animal to inhale the drug, which is convenient for the animal to accept the drug. After the drug is injected into a drug storage cavity, drug particles reach a drug inhalation cavity through a one-way valve of the respirator with an airflow breathed by an animal as a medium, and then are inhaled by the animal into its respiratory tract.

In the related prior art, a pet assistive drug delivery respirator includes a drug storage cavity and a drug inhalation cavity, where the drug inhalation cavity communicates with the drug storage cavity; a communication portion between the drug inhalation cavity and the drug storage cavity is provided with a wafer one-way valve; an exhalation hole is formed on a side wall of the drug inhalation cavity; and an exhalation one-way valve is provided at an exhaust port. During drug delivery, a drug is first injected into the drug storage cavity; when an animal inhales, the exhalation one-way valve closes the exhalation hole, and the drug enters the drug inhalation cavity from the drug storage cavity through the wafer one-way valve and finally enters a respiratory tract of the animal; and when the animal exhales, the wafer one-way valve closes the communication portion between the drug inhalation cavity and the drug storage cavity, and an airflow is discharged from the exhalation hole.

For the above-mentioned related art, the inventors believe that, after an animal inhales, there is a drug residue in the drug inhalation cavity, and the drug residue is discharged from the exhalation hole when the animal exhales, which reduces the utilization rate of the drug. Thus, it is necessary to improve the drug delivery respirator.

### SUMMARY

In order to reduce the residue of a drug in a drug inhalation cavity to increase the utilization rate of the drug, in one aspect, this application provides a one-way respiratory valve.

The one-way respiratory valve provided in this application adopts the following technical solution:

A one-way respiratory valve is provided, including a valve body, where the valve body is hollow, opposite ends of the valve body are open, a partition plate is provided at one of the opposite ends, and an inhalation hole is formed at the other one of the opposite ends; an exhalation hole is formed on a side wall of the valve body, a second unidirectional guide member is provided at the exhalation hole of the valve body, and the second unidirectional guide member is unidirectionally accessible from an inside of the valve body to an outside of the valve body; and a middle part of the partition plate protrudes towards the inhalation hole to form a first protrusion, a first unidirectional guide member is provided on the first protrusion, and the first unidirectional guide member is unidirectionally accessible from the outside of the valve body to the inside of the valve body.

In the above technical solution, the middle part of the partition plate protrudes towards the inside of the wave body to form a first protrusion, and the first unidirectional guide member is provided on the first protrusion. Compared with the wafer one-way valve, the first protrusion reduces both an internal volume of the valve body and a distance between the first unidirectional guide member and the inhalation hole, such that the residue of a drug in the valve body after inhalation is reduced, and thus the waste caused by the discharge of the residual drug through the exhalation hole during exhalation is reduced, which helps to improve the utilization rate of the drug.

Optionally, a cross-sectional area of the valve body may gradually decrease from the partition plate to the inhalation hole.

In the above technical solution, due to a guiding function, an inner wall of the tapered valve body guides an airflow from the first unidirectional guide member to the inhalation hole during inhalation, and guides an airflow from the inhalation hole to the exhalation hole during exhalation, which helps to improve the smoothness of use of the one-way respiratory valve by an animal, and also helps to reduce the residue of a drug in the valve body.

Optionally, the side wall of the valve body may protrude away from the inside of the valve body to form a second protrusion, and the exhalation hole may be located on the second protrusion.

The formation of the exhalation hole directly on the side wall of the valve body is not conducive to the outflow of an airflow during exhalation and is also likely to cause the residue of a drug in the valve body. In the above technical solution, the second protrusion is provided, and the exhalation hole is formed on the second protrusion, which facilitates the outflow of an airflow during exhalation and reduces the residue of a drug in the valve body.

Optionally, the exhalation hole may be located between a bottom and an end of the first protrusion in an axial direction of the valve body.

In the above technical solution, the exhalation hole is located between the first protrusion and the inhalation hole, which facilitates the smooth discharge of an airflow through the exhalation hole during exhalation.

Optionally, a mask may be provided at the inhalation hole outside the valve body.

In the above technical solution, the mask is replaceable and easy to use, which helps to improve the safety, health, and use convenience of the one-way respiratory valve.

Optionally, an avoidance groove may be formed inside the valve body, a plate lug may be provided on the partition plate, and the plate lug may be inserted into the avoidance groove.

In the above technical solution, the plate lug is inserted into the avoidance groove, such that the partition plate is fixed tightly, which reduces the leakage of an airflow through a gap between the partition plate and the valve body and helps to improve the utilization rate of a drug.

In another aspect, this application also provides an animal assistive drug delivery respirator provided with a one-way respiratory valve, where the one-way respiratory valve refers to the one-way respiratory valve described above; a cylinder body is detachably connected to one end of the valve body that is close to the partition plate, the cylinder body is hollow, and the cylinder body communicates with the inside of the valve body; and a cylinder cover is detachably connected to one end of the cylinder body that is away from the valve body, a drug injection hole is formed on the cylinder cover, and the drug injection hole communicates with an inside of the cylinder body.

On the above technical solution, the cylinder body and the cylinder cover are provided on the one-way respiratory valve, such that, during drug delivery for an animal, a drug is first injected into the cylinder body through the drug injection hole and then inhaled into the body through the one-way respiratory valve, which helps to control the dosage and reduce the excessive use of the drug (causing harm to animal health).

Optionally, a locking block may be provided on an outer side wall of the valve body, a locking slot may be formed on an inner wall of the cylinder body, and the locking block may be fitted with the locking slot.

In the above technical solution, the locking block is fitted with the locking slot, such that the cylinder body is tightly fixed to the wave body, which reduces the leakage of an airflow through a gap between the cylinder body and the valve body and helps to improve the utilization rate of a drug.

Optionally, a guiding slope may be formed at an edge of an inner wall of the drug injection hole that is away from the inside of the valve body.

In the above technical solution, the guiding slope is convenient for a user to provide a drug injection device into the drug injection hole, which improves the use convenience of the animal assistive drug delivery respirator provided with a one-way respiratory valve.

In summary, this application at least has the following beneficial effects:

In the present disclosure, the partition plate is provided, the middle part of the partition plate protrudes towards the inside of the wave body to form a first protrusion, and the first unidirectional guide member is provided on the first protrusion. Compared with the wafer one-way valve, the first protrusion reduces both an internal volume of the valve body and a distance between the first unidirectional guide member and the inhalation hole, such that the residue of a drug in the valve body after inhalation is reduced, and thus the waste caused by the discharge of the residual drug through the exhalation hole during exhalation is reduced, which helps to improve the utilization rate of the drug.

In the present disclosure, the cylinder body and the cylinder cover are provided on the one-way respiratory valve, such that, during drug delivery for an animal, a drug is first injected into the cylinder body through the drug injection hole and then inhaled into the body through the one-way respiratory valve, which helps to control the dosage and reduce the excessive use of the drug (causing harm to animal health).

### BRIEF DESCRIPTION OF THE DRAWINGS

FIG. 1 is an exploded view illustrating a structure of a one-way respiratory valve in an embodiment of this application;

FIG. 2 is a schematic diagram illustrating a structure of a partition plate in an embodiment of this application;

FIG. 3 is a schematic diagram illustrating a structure of a one-way respiratory plate in an embodiment of this application;

FIG. 4 is a cross-sectional view obtained along an A-A direction in FIG. 1 of this application;

FIG. 5 is an axonometric drawing illustrating an overall structure of an animal assistive drug delivery respirator provided with a one-way respiratory valve in an embodiment of this application;

FIG. 6 is a cross-sectional view obtained along a B-B direction in FIG. 5 of this application; and

FIG. 7 is an exploded view illustrating a structure of an animal assistive drug delivery respirator provided with a one-way respiratory valve in an embodiment of this application.

Reference numerals: 1: valve body; 11: partition plate; 111: first protrusion; 112: first unidirectional guide member; 12: inhalation hole; 13: exhalation hole; 131: second unidirectional guide member; 2: plate lug; 21: avoidance groove; 3: second protrusion; 4: mask; 5: cylinder body; 6: cylinder cover; 61: drug injection hole; 611: guiding slope; 62: anti-cracking slot; 63: inner wall; 7: locking slot; 71: locking block; 8: one-way respiratory plate; 81: chuck; and 82: mounting groove.

### DETAILED DESCRIPTION OF THE EMBODIMENTS

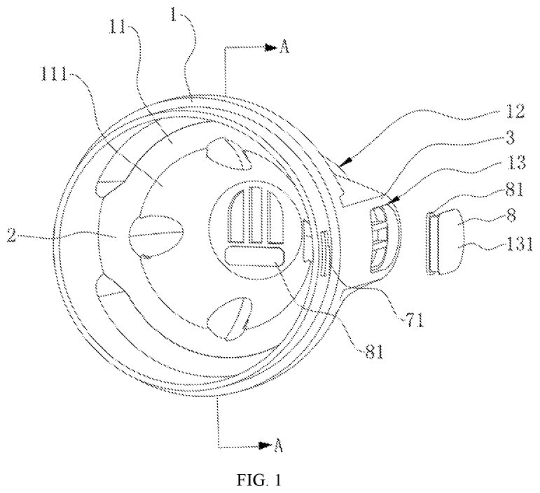

This application will be further described in detail below with reference to FIG. 1 to FIG. 7.

An embodiment of this application discloses a one-way respiratory valve.

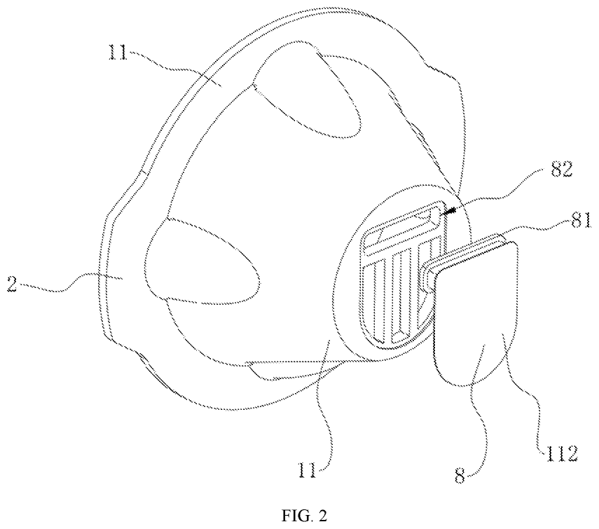

As shown in FIG. 1 and FIG. 2, the one-way respiratory valve includes a valve body 1, which can be a conical valve body 1. The valve body 1 is hollow, and two ends of the valve body 1 are open. A partition plate 11 is detachably fixed at one of the two ends that has a large cross-sectional area, and an inhalation hole 12 is formed at one of the two ends that has a small cross-sectional area. An exhalation hole 13 is formed on a side wall of the valve body 1, and a second unidirectional guide member 131 is removably fixed at the exhalation hole 13 of the valve body 1. The second unidirectional guide member 131 is unidirectionally accessible from an inside of the valve body 1 to an outside of the valve body 1. There are two exhalation holes 13 in total, and the two exhalation holes 13 are symmetrically formed on the side wall of the valve body 1. A middle part of the partition plate 11 protrudes towards an inside of the wave body 1 to form a first protrusion 111, and the first unidirectional guide member 112 is detachably fixed on the first protrusion 111. The first unidirectional guide member 112 is unidirectionally accessible from an end of the valve body 1 that has a large cross-sectional area to the inhalation hole 12.

In use, when an animal inhales, an airflow flows in from the first unidirectional guide member 112 of the partition plate 11 and flows out from the inhalation hole 12; and when an animal exhales, an airflow flows in from the inhalation hole 12 and flows out from the exhalation hole 13.

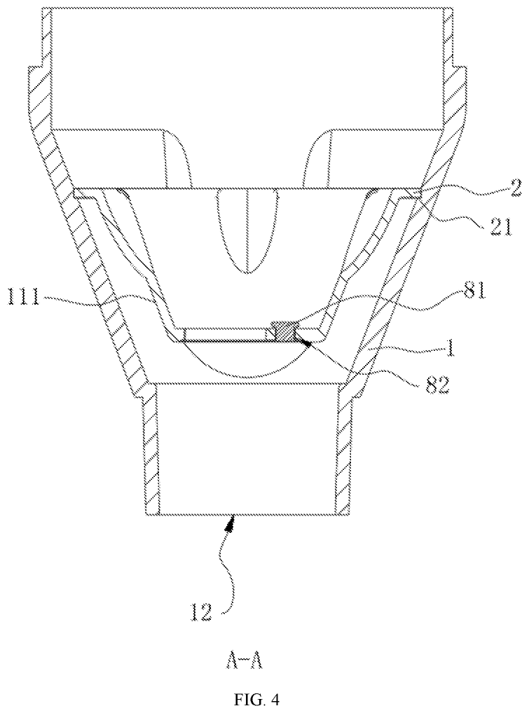

As shown in FIG. 1 and FIG. 4, in order to enable smooth outflow of an airflow from the valve body 1 through the exhalation hole 13, a side wall of the valve body 1 located at the exhalation hole 13 protrudes towards an outside of the valve body 1 to form a second protrusion 3. The second protrusion 3 corresponds to the exhalation holes 13, and the exhalation hole 13 is located on a top of the second protrusion 3. A shape of the second protrusion 3 matches a shape of the second unidirectional guide member 131, and the exhalation hole 13 is located between the first unidirectional guide member 112 and the inhalation hole 12.

As shown in FIG. 1 and FIG. 2, in this embodiment, the first unidirectional guide member 112 and the second unidirectional guide member 131 both are one-way respiratory plates 8. The one-way respiratory plate 8 is rectangular as a whole, and one end thereof in a length direction is rounded. The one-way respiratory plate 8 at the first protrusion 111 is located at a side of a top end of the first protrusion 111 that is close to the inhalation hole 12, and the one-way respiratory plate 8 at the second protrusion 3 is located at an outer side of the valve body 1. The one-way respiratory plate 8 can be made of silica gel. When the one-way respiratory plate 8 is guiding, the one-way respiratory plate 8 is opened due to the push of an airflow. When the one-way respiratory plate 8 is not guiding, the one-way respiratory plate 8 closes a corresponding inhalation hole 12 or exhalation hole 13 due to its own elasticity and the air pressure.

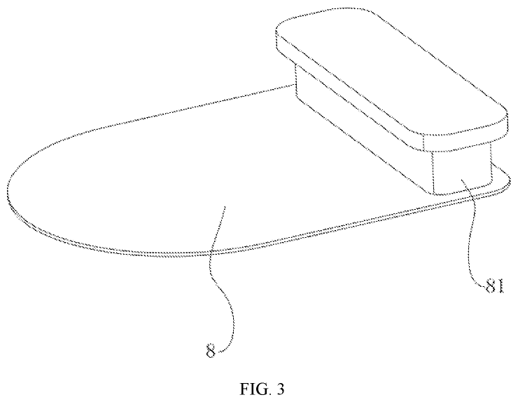

As shown in FIG. 2 and FIG. 3, a chuck 81 is provided on the one-way respiratory plate 8, the chuck 81 has a waist-round cross-section, and a cross-sectional area of an end of the chuck 81 away from the one-way respiratory plate 8 is larger than a cross-sectional area of an end of the chuck 81 close to the one-way respiratory plate 8. The chuck 81 is located at one side of the one-way respiratory plate 8 in a thickness direction, and is also located at an unrounded end of the one-way respiratory plate 8.

As shown in FIG. 1 and FIG. 2, a mounting groove 82 is formed on the first protrusion 111. The mounting groove 82 penetrates through the partition plate 11, a shape of the mounting groove 82 is similar to a cross-sectional shape of the chuck 81, and a cross-sectional area of the mounting groove 82 is smaller than a cross-sectional area of an end of the chuck 81 away from the one-way respiratory plate 8. The chuck 81 of the one-way respiratory plate 8 located at the first protrusion is inserted into the mounting groove 82. Two mounting grooves 82 are also formed respectively on two second protrusions 3, which correspond to the two one-way respiratory plates 8 on the two second protrusions 3, respectively. The chucks 81 on the two one-way respiratory plates 8 at the two second protrusions 3 are inserted into corresponding mounting grooves 82.

As shown in FIG. 1, in order to tightly fix the partition plate 11, an avoidance groove 21 is formed inside the valve body 1. A depth direction of the avoidance groove 21 is perpendicular to an axial direction of the valve body 1. The partition plate 11 is provided with a plate lug 2, and the plate lug 2 is inserted into the avoidance groove 21. Two avoidance grooves 21 are symmetrically formed inside the valve body 1, two plate lugs 2 are provided on the partition plate 11, and the plate lugs 2 are in one-to-one correspondence with the avoidance grooves 21.

When an animal inhales a drug from the inhalation hole 12, a respiratory tract can be connected or a mask 4 can be used. In this embodiment, the valve body is provided with a mask 4 at the inhalation hole, and the mask 4 is sleeved outside one end of the valve body 1 close to the inhalation hole 12.

An embodiment of this application also discloses an animal assistive drug delivery respirator provided with a one-way respiratory valve.

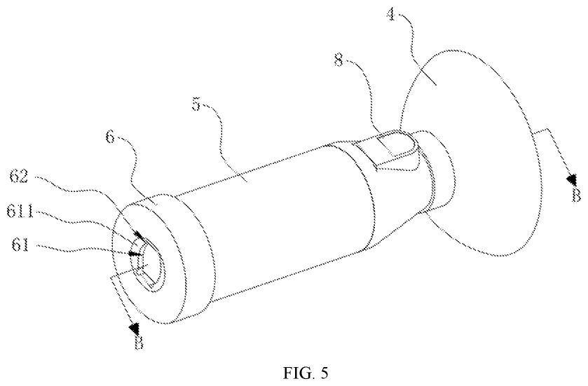

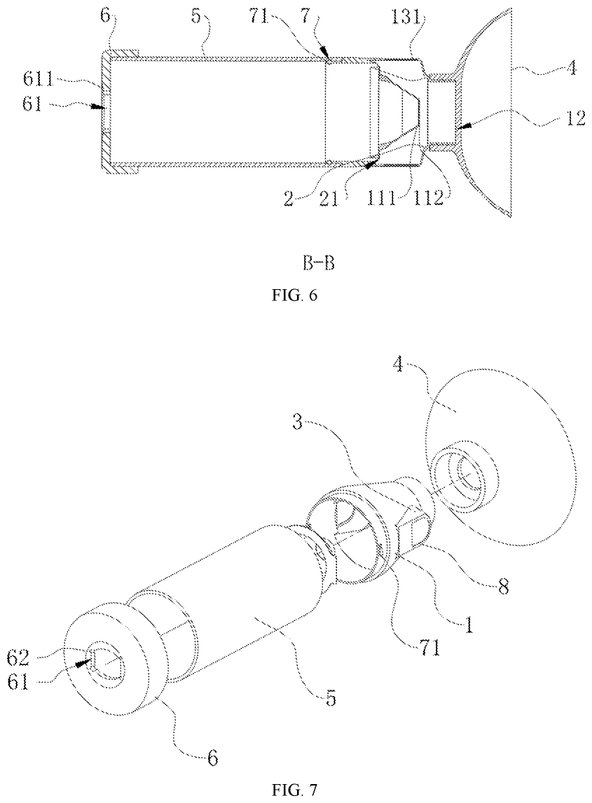

As shown in FIG. 5 and FIG. 6, the animal assistive drug delivery respirator includes the one-way respiratory valve described above. A cylinder body 5 is provided at an end of the valve body 1 close to the partition plate 11. The cylinder body 5 is in a hollow cylinder. An outer diameter of an end of the valve body 1 close to the cylinder body 5 is slightly smaller than an inner diameter of an end of the cylinder body 5 close to the valve body 1. The valve body 1 is inserted into the cylinder body 5, and the cylinder body 5 communicates with the inside of the valve body 1. An end of the cylinder body 5 away from the valve body 1 is provided with a cylinder cover 6. The cylinder cover 6 can be a rubber cover. The cylinder cover 6 is coaxially buckled on the cylinder body 5. A drug injection hole 61 is formed on the cylinder cover 6, and the drug injection hole 61 communicates with the inside of the cylinder body 5.

In use, during drug delivery for an animal, a drug is first injected into the cylinder body 5 through the drug injection hole 61, and then inhaled into the body through the one-way respiratory valve.

As shown in FIG. 6 and FIG. 7, in order to improve the tightness of the fixation of the cylinder body 5 to the valve body 1, a locking slot 7 is formed on an inner wall of the cylinder body 5 close to the valve body 1. There can be a plurality of locking slots 7. Two locking slots 7 are preferred in this embodiment, which are oppositely arranged on the inner wall of the cylinder body 5. A locking block 71 is formed on an outer wall of the valve body 1 close to the cylinder body 5. There are two locking blocks 71, which are in one-to-one correspondence with the locking slots 7. When the valve body 1 is fixed to the cylinder body 5, the locking blocks 71 are inserted into corresponding locking slots 7.

As shown in FIG. 5 and FIG. 6, in order to facilitate the injection of a drug into the cylinder body 5 by a user, a guiding slope 611 is formed at the drug injection hole 61 outside the cylinder cover 6 and along an edge of an inner wall of the drug injection hole 61.

Since the cylinder cover 6 is a rubber cover, the drug injection hole 61 is formed by sequentially connecting a plurality of (four inner walls are preferred in this embodiment) inner walls 63 end-to-end. Repeated plugging and unplugging of a drug injection device at the drug injection hole 61 is easy to make junctions among the inner walls 63 of the drug injection hole 61 cracked, thereby causing damage to the cylinder cover 6. Therefore, an anti-cracking slot 62 is formed at each of the junctions among the inner walls 63 of the drug injection hole 61 along an axial direction of the drug injection hole 61. The anti-cracking slot 62 helps to reduce damage to the cylinder cover 6.

Implementation principle of this embodiment: During drug delivery for an animal, a worker first injects a drug into the cylinder body 5 through the drug injection hole 61. When the animal inhales, due to the unidirectional guiding of the first unidirectional guide member 112 and the second unidirectional guide member 131, an inhaled airflow drives the drug in the cylinder body 5 into the valve body 1, and then the drug is inhaled by the animal from the inhalation hole 12. When the animal exhales, due to the unidirectional guiding of the first unidirectional guide member 112 and the second unidirectional guide member 131, an exhaled airflow is discharged out of the valve body 1 through the inhalation hole 12 and the exhalation hole 13.

The above are preferred embodiments of this application, but the protection scope of this application is not limited thereto. Therefore, all equivalent changes made in accordance with the structure, shape, and principle of this application shall fall within the protection scope of this application.

What is claimed is:

1. A one-way respiratory valve, comprising a valve body, wherein
the valve body is hollow, opposite ends of the valve body are open, a partition plate is provided at a first end of the opposite ends, and an inhalation hole is formed at a second end of the opposite ends;
an exhalation hole is formed on a side wall of the valve body, a second unidirectional guide member is provided at the exhalation hole of the valve body, and the second unidirectional guide member is unidirectionally accessible from an inside of the valve body to an outside of the valve body; and
a middle part of the partition plate protrudes towards the inhalation hole to form a first protrusion, a first unidirectional guide member is provided on the first protrusion, and the first unidirectional guide member is unidirectionally accessible from the outside of the valve body to the inside of the valve body.

2. The one-way respiratory valve according to claim 1, wherein
a cross-sectional area of the valve body gradually decreases from the partition plate to the inhalation hole.

3. The one-way respiratory valve according to claim 1, wherein
the side wall of the valve body protrudes away from the inside of the valve body to form a second protrusion, and the exhalation hole is located on the second protrusion.

4. The one-way respiratory valve according to claim 1, wherein
the exhalation hole is located between a bottom of the first protrusion and an end of the first protrusion in an axial direction of the valve body.

5. The one-way respiratory valve according to claim 1, wherein
a mask is provided at the inhalation hole outside the valve body.

6. The one-way respiratory valve according to claim 1, wherein
an avoidance groove is formed inside the valve body, a plate lug is provided on the partition plate, and the plate lug is inserted into the avoidance groove.

7. An animal assistive drug delivery respirator, comprising the one-way respiratory valve according to claim 1; wherein
a cylinder body is detachably connected to one end of the valve body, the end of the valve body is close to the partition plate, the cylinder body is hollow, and the cylinder body communicates with the valve body; and
a cylinder cover is detachably connected to one end of the cylinder body, the end of the cylinder body is away from the valve body, a drug injection hole is formed on the cylinder cover, and the drug injection hole communicates with an inside of the cylinder body.

8. The animal assistive drug delivery respirator according to claim 7, wherein
a locking block is provided on an outer side wall of the valve body, a locking slot is formed on an inner wall of the cylinder body, and the locking block is fitted with the locking slot.

9. The animal assistive drug delivery respirator according to claim 7, wherein
a guiding slope is formed at an edge of an inner wall of the drug injection hole, and the edge is away from the inside of the valve body.

10. The animal assistive drug delivery respirator according to claim 7, wherein
a cross-sectional area of the valve body gradually decreases from the partition plate to the inhalation hole.

11. The animal assistive drug delivery respirator according to claim 7, wherein
the side wall of the valve body protrudes away from the inside of the valve body to form a second protrusion, and the exhalation hole is located on the second protrusion.

12. The animal assistive drug delivery respirator according to claim 7, wherein
the exhalation hole is located between a bottom of the first protrusion and an end of the first protrusion in an axial direction of the valve body.

13. The animal assistive drug delivery respirator according to claim 7, wherein
a mask is provided at the inhalation hole outside the valve body.

14. The animal assistive drug delivery respirator according to claim 7, wherein
an avoidance groove is formed inside the valve body, a plate lug is provided on the partition plate, and the plate lug is inserted into the avoidance groove.
